(19) United States
(12) Patent Application Publication
(10) Pub. No.: US 2012/0065226 A1
(43) Pub. Date: Mar. 15, 2012

(54) SALTS OF TIOTROPIUM WITH 10-CAMPHORSULFONIC ACID

(75) Inventors: Helena Modrzejewska, Warszawa (PL); Zbigniew Majka, Lubzina (PL); Magdalena Dziedzic-Lysiak, Bogoria (PL)

(73) Assignee: ADAMED SP Z O.O., Czosnow k/Warszawy (PL)

(21) Appl. No.: 13/320,627

(22) PCT Filed: May 7, 2010
(86) PCT No.: PCT/EP2010/056279

§ 371 Date: Nov. 15, 2011

(30) Foreign Application Priority Data
May 19, 2009 (PL) P-388070

(51) Int. Cl. A61K 31/439; A61P 11/00; C07D 491/08

(52) U.S. Cl. 514/302; 546/91

(57) ABSTRACT

The present invention relates to novel tiotropium salts with 10-camphorsulphonic acid of formula (I), its optical isomers or their mixtures, their monohydrates, their anhydrous crystalline forms and processes for the preparation thereof, as well as to pharmaceutical compositions containing the same and to the use of novel salts for the treatment of respiratory tract diseases.

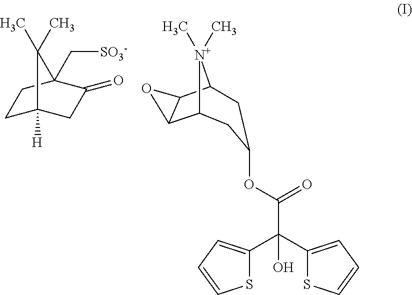

### FIELD OF THE INVENTION

The present invention relates to novel tiotropium salts, as well as its hydrates and anhydrous forms, processes for the preparation thereof and pharmaceutical compositions containing the same. The novel salts are useful for the treatment of respiratory tract diseases, such as asthma and chronic obstructive pulmonary disease (COPD).

### STATE OF THE ART

Tiotropium is the International Non-proprietary Name (INN) of quaternary ammonium derivative having the chemical name: (1α,2β,4β,5α,7β)-7-[(hydroxy-di-2-thienylacetyl)oxy]-9,9-dimethyl-3-oxa-9-azoniatricyclo[3.3.1.0^{2,4}]nonan and the following chemical structure:

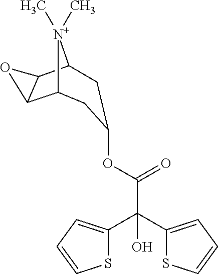

The compound is a synthetic anticholinergic, used in the treatment of asthma and chronic obstructive pulmonary disease (COPD). The compound, its activity and usefulness in medicine were for the first time disclosed in WO 91/04252.

In the publication WO 91/04252 mentioned above two tiotropium salts bromide and methanesulphonate, and processes for the preparation thereof are disclosed.

Crystalline tiotropium bromide monohydrate is an active pharmaceutical ingredient of commercial pharmaceutical compositions: capsules containing a powder for inhalation (Spiriva®) and solution for inhalation (Spiriva Respimat®).

Crystalline tiotropium bromide monohydrate is disclosed in WO 02/30928 as a chemically and physically stable form of that salt.

Further crystalline forms of tiotropium bromide are disclosed in WO 03/000265, where crystalline tiotropium bromide obtained by drying of monohydrate are described; in WO 05/042527, where anhydrous form of tiotropium bromide with improved stability in the presence of moisture were described; in WO 06/117299 and WO 06/117300, where crystalline solvates of tiotropium bromide and non-solvated form thereof were disclosed. Publications WO 07/075838 and WO 07/075858 disclose crystalline solvates of tiotropium bromide, inter alia solvates with alcohols and with acetic acid, as well as amorphous form of tiotropium bromide.

The process for the preparation of tiotropium bromide and methanesulfonate comprises direct quaternization of di-(2-thienyl)glycolic acid scopine ester (9-methyl-3-oxa-9-azatricyclo[3.3.1.0^{2,4}]non-7-yl hydroxy(di-2-thienyl)acetate) with methyl bromide or methyl methanesulfonate, respectively (WO 91/04252 and WO 05/042526).

Apart from bromide, further tiotropium salts that are disclosed and characterized in terms of their physicochemical properties are methane-sulphonate, methylsulphate, chloride and iodide (WO 05/042526), benzoate, saccharate, and p-toluenesulphonate (WO 05/042528), bicarbonate, benzenesulphonate, trifluoromethanesulphonate, salicylate, hydrogensulphate, ethanedisulphonate, xinafoate, fumarate, malate, succinate, malonate, tartrate, and oxalate (WO 06/134021). Co-crystal of tiotropium bromide with urea characterized as relatively stable towards the influence of moisture and humidity is disclosed in WO 08/058968.

Prior art is silent about any particular advantages of those various other tiotropium salts over tiotropium bromide, especially in terms of their improved chemical or physical stability or suitability for formulation into pharmaceutical preparations. Moreover, tiotropium salts other than halogenides, arylsulphonates and alkylsulphonates cannot be prepared directly by quaternization and are usually manufactured by anion exchange using tiotropium halogenides as starting materials. This results in a longer manufacturing process and losses in the yield of a final salt product.

Active pharmaceutical substance must meet very strict requirements regarding both chemical and physical stability of the substance itself, and its stability during manufacturing and storage of pharmaceutical preparations. Tiotropium bromide and crystalline forms thereof, especially crystalline tiotropium bromide monohydrate, are indicated in the prior art as preferable due to physicochemical properties thereof and due to suitability for pharmaceutical formulation.

However, according to the prior art, tiotropium bromide is simultaneously susceptible to physical transformations, particularly in the presence of moisture. Due to the necessity to prevent the physical and chemical transformations of tiotropium bromide the use of capsules (inhalettes) with reduced moisture content is proposed in WO 02/098874, and the use of containers providing high barrier towards moisture is proposed in WO 05/053644 and WO 05/053646.

Apart from chemical stability during storage, resistance towards the influence of moisture, temperature, oxidation, phenomena of polymorphism and pseudopolymorphism may be of particular importance, especially for powder compositions. For pharmaceutical substances exhibiting extensive polymorphism, such as tiotropium bromide as shown in above mentioned publications, there is a risk of polymorphic transformations both during its manufacturing and storage, and during formulation and storage of the finished pharmaceutical product. Due to differences, for example in solubility, between crystalline modifications of the same compound, transformation of one form into another during manufacturing or storage may result in changes of dissolution rate, bioavailability, and in extreme cases in loss of biological activity of an active substance. For these reasons phenomenon of polymorphism of tiotropium bromide, especially when it is a result of spontaneous process dependent for example on storage conditions, is undesirable.

### AIM OF THE INVENTION

The aim of the present invention is therefore to provide a novel, physically and chemically stable tiotropium salt with pharmaceutically acceptable counterion which can be obtained with high purity in a high-yield and a technologically non-complicated process and which is suitable for manufacturing of stable pharmaceutical compositions not requiring particular precautionary measures during manufacture, storage and use.

The aim has been achieved by inventing novel tiotropium salts with 10-camphorsulphonic acid and its optical isomers.

### DESCRIPTION OF THE DRAWINGS

FIG. 1 shows an X-ray powder diffraction pattern (XRPD) of crystalline anhydrous tiotropium salt with 1R-(−)-10-camphorsulphonic acid.

FIG. 2 shows an X-ray powder diffraction pattern (XRPD) of crystalline anhydrous s tiotropium salt with 1S-(+)-10-camphorsulphonic acid.

FIG. 3 shows an X-ray powder diffraction pattern (XRPD) of crystalline anhydrous tiotropium salt with (±)-10-camphorsulphonic acid.

FIG. 4 shows an X-ray powder diffraction pattern (XRPD) of crystalline monohydrate of tiotropium salt with 1R-(−)-10-camphorsulphonic acid.

FIG. 5 shows an X-ray powder diffraction pattern (XRPD) of crystalline monohydrate of tiotropium salt with 1S-(+)-10-camphorsulphonic acid.

### DETAILED DESCRIPTION OF THE INVENTION

The present invention relates to novel salts of tiotropium, that is (1α,2β,4β,5α,7β)-7-[(hydroxydi-2-thienylacetyl)oxy]-9,9-dimethyl-3-oxa-9-azoniatricyclo[3.3.1.0^{2,4}]nonane, with 10-camphorsulphonic acid and with its single optical isomers or mixtures thereof.

In particular, the invention relates to tiotropium salt with racemic (±)-10-camphorsulphonic acid of formula (I):

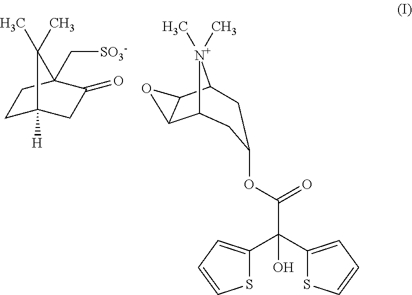

The present invention relates also to tiotropium salts with optical isomers of 10-camphorsulphonic acid: a salt with 1R-(+10-camphorsulphonic acid having the structure corresponding to formula (II):

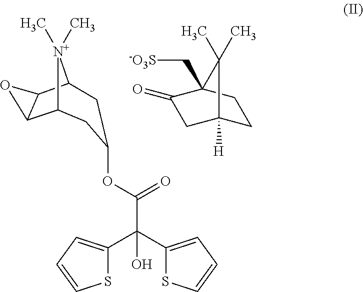

and a salt with 1S-(+)-10-camphorsulphonic acid having the structure corresponding to formula (III):

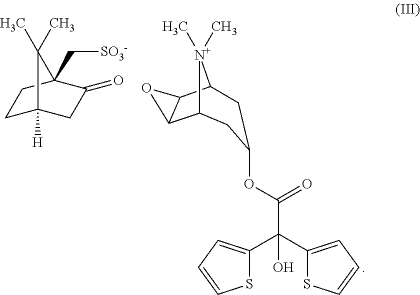

The present invention relates also to a monohydrate form of tiotropium salt with 1R-(−)-10-camphorsulphonic acid, and to a monohydrate form of tiotropium salt with 1S-(+)-10-camphorsulphonic acid.

In a further aspect the present invention relates to a process for the preparation of tiotropium salt with 10-camphorsulphonic acid which comprises quaternization of 9-methyl-3-oxa-9-azatricyclo[3.3.1.0^{2,4}]non-7-yl hydroxy(di-2-thienyl)acetate in an organic solvent with methyl 10-camphorsulphonate, followed by isolation of the tiotropium salt.

Preferably, a molar ratio of 9-methyl-3-oxa-9-azatricyclo[3.3.1.0^{2,4}]non-7-yl hydroxy(di-2-thienyl)acetate to methyl 10-camphorsulphonate is in the range from 1:1 to 1:5.

Solvents suitable for the reaction may be selected from a group consisting of aromatic hydrocarbons, such as toluene; alcohols, such as methanol, ethanol, and n-butanol; esters, such as ethyl acetate; ketones, such as acetone, methyl-ethyl ketone, diisopropyl ketone, and methyl-tert-butyl ketone; ethers such as diethyl ether, tetrahydrofuran, and methyl-tert-butyl ether; halogenated hydrocarbons, such as methylene chloride; nitriles, such as acetonitrile, or mixtures thereof.

In a preferred embodiment, the above process comprises quaternization of 9-methyl-3-oxa-9-azatricyclo[3.3.1.0^{2,4}]non-7-yl hydroxy(di-2-thienyl)acetate in an acetonitrile/methylene chloride mixture. After completion of the reaction, precipitated solid tiotropium salt is isolated directly from the reaction mixture by filtration, or after the concentration of the reaction mixture an organic solvent is added to the residue, followed by trituration and then filtration of the crystalline product. After filtration the isolated product can be washed with the organic solvent. The product is then dried, preferably under reduced pressure. The crystalline anhydrous salt with advantageous properties is a direct product of the above process.

In one specific embodiment of the process according to the invention, said process comprises quaternization of 9-methyl-3-oxa-9-azatricyclo[3.3.1.0^{2,4}]-non-7-yl hydroxy(di-2-thienyl)acetate with methyl 1R-(−)-10-camphorsulphonate in the organic solvent as described above, to obtain anhydrous tiotropium salt with 1R-(−)-10-camphorsulphonic acid of formula (II). Preferably, the molar ratio of 9-methyl-3-oxa-9-azatricyclo[3.3.1.0^{2,4}]non-7-yl hydroxy(di-2-thienyl)-acetate to methyl 1R-(−)-10-camphorsulphonate is in the range from 1:1 to 1:5.

Advantageously, the salt of formula (II) is prepared in a crystalline anhydrous form. After completion of the reaction, precipitated crystalline product is isolated directly from the reaction mixture by filtration or, after concentration of the reaction mixture, an organic solvent is added to the residue, followed by trituration and then filtration of the crystalline product. After filtration the isolated product can be washed with the organic solvent. The product is then dried, preferably under reduced pressure. Preferably, the organic solvent for trituration is methylene chloride or acetone. The crystalline anhydrous salt with advantageous properties is a direct product of above process.

As an example, in a preferred embodiment, to 9-methyl-3-oxa-9-azatricyclo-[3.3.1.0^{2,4}]non-7-yl hydroxy(di-2-thienyl)acetate in an acetonitrile/methylene chloride mixture, methyl 1R-(+10-camphorsulphonate dissolved in acetonitrile is added. Crystalline product which precipitates after concentration of the reaction mixture by evaporation is triturated in methylene chloride, isolated by filtration and then dried under reduced pressure.

The invention relates therefore also to the crystalline anhydrous tiotropium salt with 1R-(−)-10-camphorsulphonic acid.

In particular, the invention relates to such salt showing characteristic peaks in the X-ray powder diffraction (XRPD) pattern expressed in two theta degrees at 5.4, 16.3, 16.9, 17.1, 17.2, 18.7, 21.2, and 22.4 (±0.2θ).

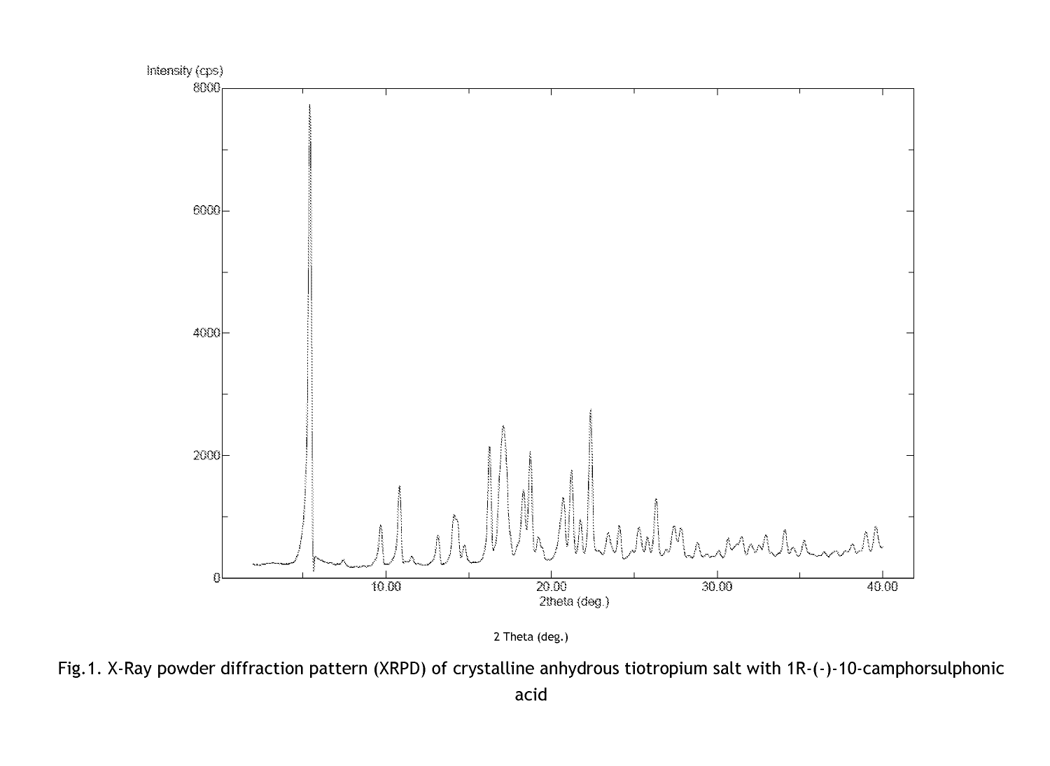

In particular, the crystalline anhydrous tiotropium salt with 1R-(−)-10-camphorsulphonic acid of the invention exhibits XRPD pattern as shown in FIG. 1

In the second specific embodiment of the process according to the invention, said process comprises quaternization of 9-methyl-3-oxa-9-azatricyclo-[3.3.1.0^{2,4}]non-7-yl hydroxy(di-2-thienyl)acetate in the organic solvent as described above with methyl 1S-(+)-10-camphorsulphonate, to obtain anhydrous tiotropium salt with 1S-(+)-10-camphorsulphonic acid of formula (III). Preferably, the molar ratio of 9-methyl-3-oxa-9-azatricyclo[3.3.1.0^{2,4}]non-7-yl hydroxy(di-2-thienyl)acetate to methyl 1S-(+)-10-camphorsulphonate is in the range from 1:1 to 1:5.

Advantageously, the salt of formula (III) is obtained in a crystalline anhydrous form. After completion of the reaction, precipitated crystalline product is isolated directly from the reaction mixture by filtration or, after concentration of the reaction mixture, an organic solvent is added to the residue, followed by trituration and then filtration of the crystalline product. After filtration the isolated product can be washed with the organic solvent. The product is then dried, preferably under reduced pressure. Preferably, trituration is performed using methylene chloride or acetone. The crystalline anhydrous salt with advantageous properties is a direct product of above process.

As an example, in a preferred embodiment, to 9-methyl-3-oxa-9-azatricyclo[3.3.1.0^{2,4}]non-7-yl hydroxy(di-2-thienyl)acetate in a mixture of acetonitrile and methylene chloride, methyl 1S-(+)-10-camphorsulphonate dissolved in acetonitrile is added. Crystalline product which precipitates after concentration of the reaction mixture by evaporation is triturated in methylene chloride, isolated by filtration and then dried under reduced pressure.

The invention relates therefore also to crystalline anhydrous tiotropium salt with 1S-(+)-10-camphorsulphonic acid.

In particular, the invention relates to such a salt showing characteristic peaks on the X-ray powder diffraction pattern expressed in two theta degrees at 5.6, 16.4, 17.0, 17.2, 17.3, 18.9, 21.3, and 22.5 (±0.2θ).

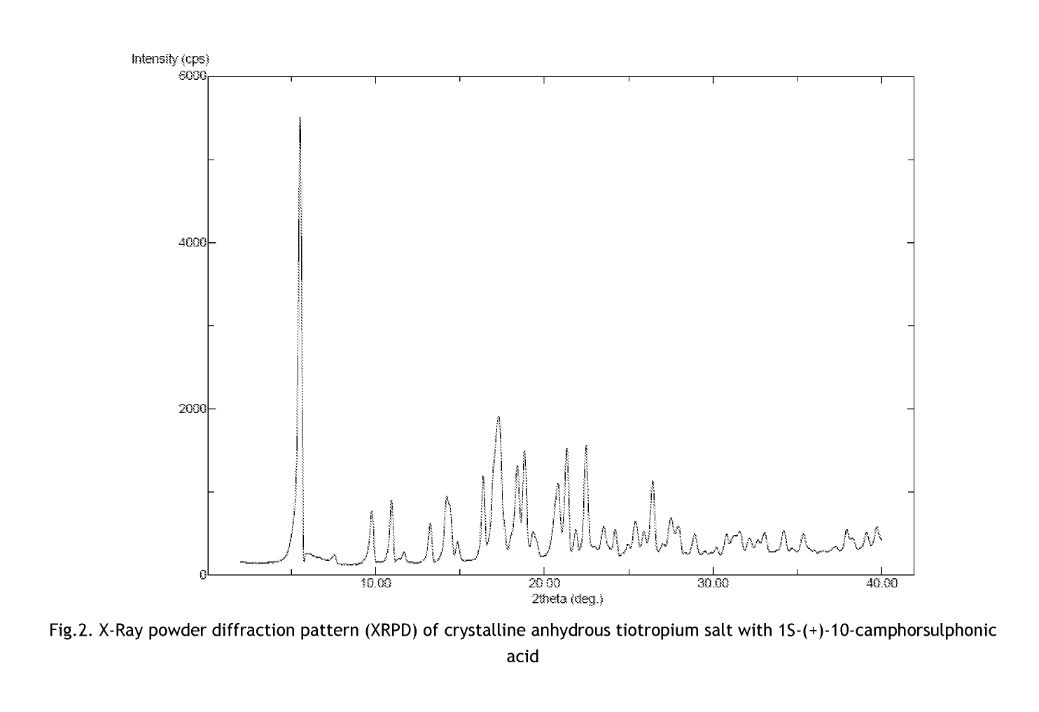

In particular, the crystalline anhydrous tiotropium salt with 1S-(+)-10-camphorsulphonic acid of the invention exhibits XRPD pattern as shown in FIG. 2.

As it can be seen from comparison of FIG. 1 and FIG. 2, crystalline anhydrous salts of tiotropium with 1R-(−)-10-camphorsulphonic acid and with 1S-(+)-10-camphorsulphonic acid have substantially identical X-ray powder diffraction patterns. These salts differ, however, in their respective optical rotation values ([α]_{D}²⁰), which may serve for the purpose of their differentiation.

In the third specific embodiment of the process according to the invention, said process comprises quaternization of 9-methyl-3-oxa-9-azatricyclo[3.3.1.0^{2,4}]-non-7-yl hydroxy(di-2-thienyl)acetate in the organic solvent as described above with methyl (±)-10-camphorsulphonate, to obtain anhydrous tiotropium salt with (±)-10-camphorsulphonic acid of formula (I). Preferably, the molar ratio of 9-methyl-3-oxa-9-azatricyclo[3.3.1.0^{2,4}]non-7-yl hydroxy(di-2-thienyl)acetate to methyl (±)-10-camphorsulphonate in quaternization reaction is in the range from 1:1 to 1:5.

Advantageously, the salt of formula (I) is prepared in the crystalline anhydrous form. After completion of the reaction, precipitated crystalline product is isolated directly from the reaction mixture by filtration or, after concentration of reaction mixture, an organic solvent is added to the residue, followed by trituration and then filtration of the crystalline product. After filtration the isolated product can be washed with the organic solvent. The product is then dried, preferably under reduced pressure. Preferably, trituration is performed using methylene chloride or acetone. The crystalline anhydrous salt with advantageous properties is a direct product of above process.

As an example, in preferred embodiment, to 9-methyl-3-oxa-9-azatricyclo[3.3.1.0^{2,4}]non-7-yl hydroxy(di-2-thienyl)acetate in a mixture of acetonitrile and methylene chloride, methyl (±)-10-camphorsulphonate dissolved in acetonitrile is added. Crystalline product precipitated after concentration of the reaction mixture by evaporation is triturated in acetone, isolated by filtration and then dried under reduced pressure.

The invention relates therefore also to the crystalline anhydrous tiotropium salt with (±)-10-camphorsulphonic acid.

In particular, the invention relates to such a salt showing characteristic peaks in the X-ray powder diffraction pattern expressed in two theta degrees at 5.4, 15.4, 20.7, and 23.4 (±0.2θ).

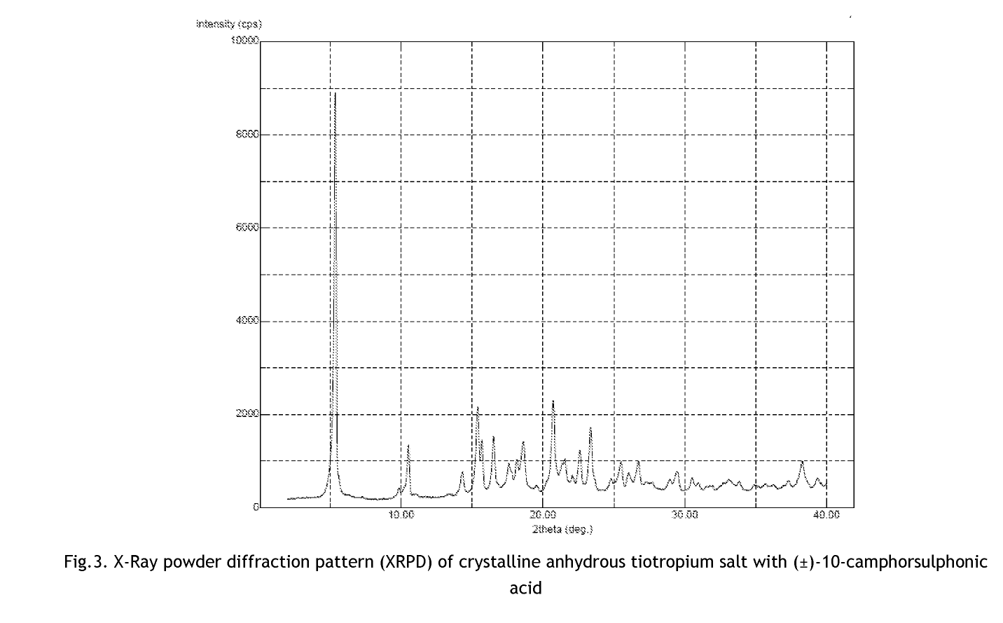

In particular, the crystalline anhydrous tiotropium salt with (±)-10-camphorsulphonic acid of the invention exhibits XRPD pattern as shown in FIG. 3.

The present invention relates also to a process for the preparation of a crystalline monohydrate of tiotropium salt with 10-camphorsulphonic acid, which comprises quaternization of 9-methyl-3-oxa-9-azatricyclo[3.3.1.0^{2,4}]non-7-yl hydroxy(di-2-thienyl) acetate in an organic solvent with methyl 10-camphorsulphonate, dissolution of the tiotropium salt isolated as described above in water, preferably at elevated temperature, and then cooling the aqueous solution. After cooling of the aqueous solution, the crystalline monohydrate of the tiotropium salt precipitates. The crystals formed are isolated by conventional techniques. Preferably, the molar ratio of 9-methyl-3-oxa-9-azatricyclo[3.3.1.0^{2,4}]non-7-yl hydroxy(di-2-thienyl)acetate to methyl 10-camphorsulphonate in the quaternization reaction is in the range from 1:1 to 1:5.

Solvents suitable for the quaternization are as described above.

In the preferred embodiment of the above process, 9-methyl-3-oxa-9-azatricyclo[3.3.1.0^{2,4}]non-7-yl hydroxy(di-2-thienyl)acetate is subjected to quaternization in an acetonitrile/methylene chloride mixture, precipitated product is isolated directly by filtration of the reaction mixture or, after concentration of the reaction mixture, an organic solvent is added to the residue, followed by trituration and then filtration of the crystalline product. After filtration the isolated product can be washed the organic solvent and/or optionally dried. The product thus obtained is subsequently dissolved in water, preferably at elevated temperature, then the solution is cooled, precipitated crystalline product is filtered off and dried, preferably under reduced pressure.

Dissolution in water is performed preferably with stirring at a temperature in the range 40-90° C., and more preferably in the range 70-80° C. The amount of water is 100-400 ml, preferably 250-350, per 100 g of the anhydrous salt.

The aqueous solution may be allowed to cool to room temperature spontaneously; cooling media, such as ice/water bath, may also be used. Obtained suspension of crystals may be additionally cooled to temperature in the range 5-15° C. and stirred at that temperature for 1-3 h, preferably for 1.5 h. Crystals of monohydrate are preferably isolated by filtration or centrifugation. The obtained crystalline product may be washed with water or an organic solvent, preferably with water. The isolated precipitate is then dried, advantageously at elevated temperature and under reduced pressure.

Content of water in the final product is in a range 2.7-3.0%, preferably it is 2.8% (analysis by Karl Fischer titration (KF)).

In one specific embodiment of the process according to the invention, 9-methyl-3-oxa-9-azatricyclo[3.3.1.0^{2,4}]non-7-yl hydroxy(di-2-thienyl)acetate in the organic solvent as described above is subjected to quaternization with methyl 1R-(−)-10-camphorsulphonate in a manner as described above, isolated product is dissolved in water, preferably at elevated temperature, and aqueous solution is then cooled to obtain crystalline monohydrate of tiotropium salt with 1R-(−)-10-camphorsulphonic acid. The crystalline monohydrate precipitates after cooling the aqueous solution. The crystals formed are isolated by conventional techniques. Preferably, the molar ratio of 9-methyl-3-oxa-9-azatricyclo[3.3.1.0^{2,4}]non-7-yl hydroxy(di-2-thienyl)acetate to methyl 1R-(−)-10-camphorsulphonate in the quaternization reaction is in the range from 1:1 to 1:5.

In the preferred embodiment of the above process, after completion of the quaternization reaction, solid product is isolated by filtration or, after concentration of reaction mixture by evaporation, by adding to the residue of an organic solvent, triturating and then filtering off. After filtration the isolated product can be washed the organic solvent and/or optionally dried. The triturating is preferably carried out in acetone. The product thus obtained is subsequently dissolved in water, preferably at elevated temperature, then the solution is cooled, precipitated crystalline product is filtered off and dried, preferably under reduced pressure.

Dissolution in water is performed preferably with stirring at a temperature in the range 40-90° C., and more preferably in the range 70-80° C. The amount of water is 100-400 ml, preferably 250-350, per 100 g of the anhydrous salt.

The aqueous solution may be allowed to cool to room temperature spontaneously; cooling media, such as ice/water bath, may also be used. Obtained suspension of crystals may be additionally cooled to temperature in the range 5-15° C. and stirred at that temperature for 1-3 h, preferably for 1.5 h. Crystals of monohydrate are preferably isolated by filtration or centrifugation. The obtained crystalline product may be washed with water or an organic solvent, preferably with water. The isolated precipitate is then subjected to drying, advantageously at elevated temperature and under reduced pressure.

Content of water in the final product is in the range of 2.7-3.0%, preferably 2.8% (analysis by Karl Fischer titration (KF)).

Crystalline monohydrate of tiotropium salt with 1R-(−)-10-camphorsulphonic acid having specific properties is a direct product of the above process.

The invention relates therefore also to the crystalline monohydrate of tiotropium salt with 1R-(−)-10-camphorsulphonic acid.

In particular, the invention relates to such a salt showing characteristic peaks on the X-ray powder diffraction pattern expressed in two theta degrees at 8.8, 15.4, 16.8, 17.5, 21.8, 25.4, and 27.6 (±0.2θ).

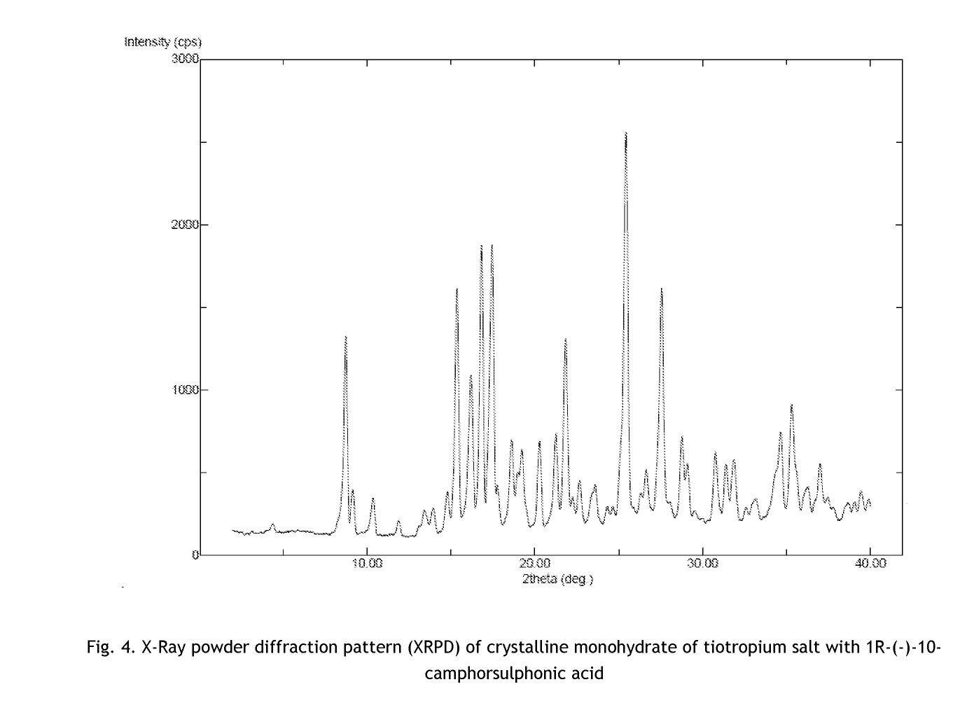

In particular, the crystalline monohydrate form of tiotropium salt with 1R-(−)-10-camphorsulphonic acid of the invention exhibits XRPD pattern as shown in FIG. 4.

In the second specific embodiment of the process according to the invention, 9-methyl-3-oxa-9-azatricyclo[3.3.1.0^{2,4}]non-7-yl hydroxy(di-2-thienyl)acetate in the organic solvent as described above is subjected to quaternization with methyl 1S-(+)-10-camphorsulphonate in a manner as described above, isolated product is dissolved in water, preferably at elevated temperature, and the aqueous solution is then cooled, to obtain crystalline monohydrate of tiotropium salt with 1S-(+)-10-camphorsulphonic acid. The crystalline monohydrate precipitates after cooling the aqueous solution. The crystals formed are isolated by conventional techniques. Preferably, a molar ratio of 9-methyl-3-oxa-9-azatricyclo[3.3.1.0^{2,4}]non-7-yl hydroxy(di-2-thienyl)acetate to methyl 1S-(+)-10-camphorsulphonate used in quaternization reaction is in the range from 1:1 to 1:5.

In the preferred embodiment of the above process, after completion of the quaternization reaction, precipitated product is isolated directly by filtration of the reaction mixture or, after concentration of the reaction mixture, an organic solvent is added to the residue, followed by trituration and then filtration of the crystalline product. After filtration the isolated product can be washed the organic solvent and/or optionally dried. Trituration is preferably carried out in acetone. The product thus obtained is subsequently dissolved in water, preferably at elevated temperature, then the solution is cooled, precipitated crystalline product thus obtained is filtered off and dried, preferably under reduced pressure.

Dissolution in water is performed preferably with stirring at a temperature in the range 40-90° C., and more preferably in the range 70-80° C. The amount of water is 100-400 ml, preferably 250-350, per 100 g of the anhydrous salt.

The aqueous solution may be allowed to cool to room temperature spontaneously; cooling media, such as ice/water bath, may also be used. Obtained suspension of crystals may be additionally cooled to a temperature in the range 5-15° C. and stirred at that temperature for 1-3 h, preferably for 1.5 h. Crystals of monohydrate are preferably isolated by filtration or centrifugation. The obtained crystalline product may be washed with water or an organic solvent, preferably with water. The isolated precipitate is then subjected to drying, advantageously at elevated temperature and under reduced pressure.

Content of water in the final product is in a range 2.7-3.0%, preferably it is 2.8% (analysis by Karl Fischer titration (KF)).

Crystalline monohydrate of tiotropium salt with 1S-(+)-10-camphorsulphonic acid having specific properties is a direct product of the above process.

The invention relates therefore also to the crystalline monohydrate form of tiotropium salt with 1S-(+)-10-camphorsulphonic acid.

In particular, the invention relates to such a salt showing characteristic peaks on the X-ray powder diffraction pattern expressed in two theta degrees at 9.0, 15.6, 17.0, 17.7, 22.0, 25.6, and 27.8 (±0.2θ).

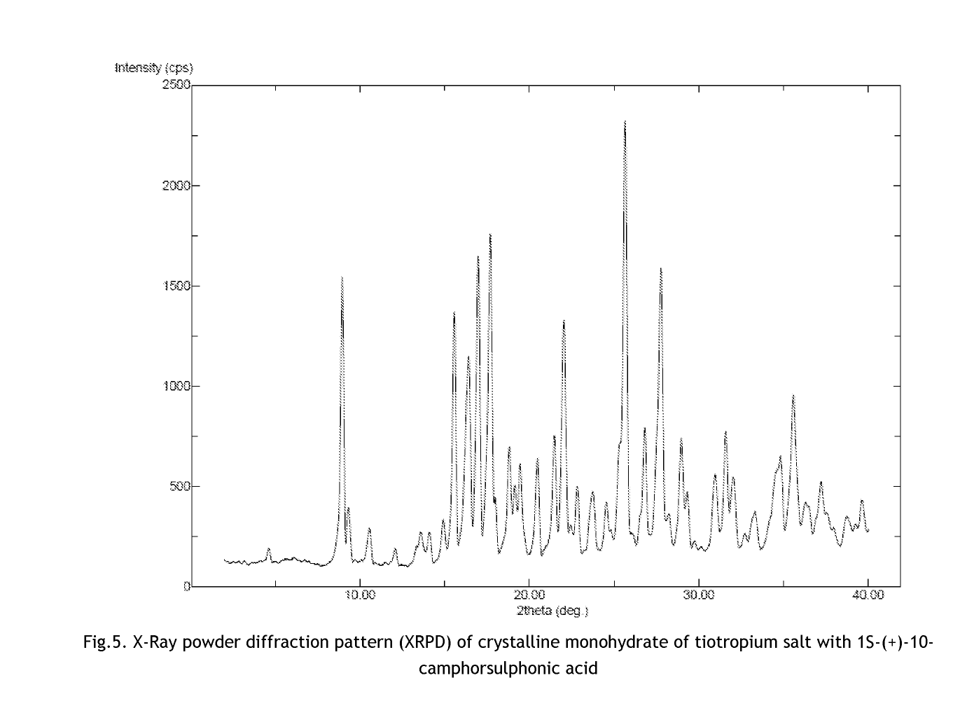

In particular, the crystalline monohydrate form of tiotropium salt with 1S-(+)-10-camphorsulphonic acid of the invention exhibits XRPD pattern as shown in FIG. 5.

As it can be seen from comparison of FIG. 4 and FIG. 5, crystalline monohydrates of the salts of tiotropium with 1R-(−)-10-camphorsulphonic acid and with 15-(+)-10-camphorsulphonic acid have substantially identical X-ray powder diffraction patterns. These salts differ, however, in their respective optical rotation values ([α]_{D}²⁰), which may serve for the purpose of their differentiation.

The process of preparation of novel salts and monohydrates thereof is efficient, allows to obtain directly crystalline products of very high purity. Crystalline monohydrates are obtained virtually free from residual organic solvents, as confirmed by GC analysis. Moreover, there is no need to use methyl 10-camphorsulphonate or a single optical isomer thereof in such a considerable molar excess in relation to 9-methyl-3-oxa-9-azatricyclo[3.3.1.0^{2,4}]non-7-yl hydroxy(di-2-thienyl) acetate and to carry out the reaction in a tightly closed vessel, as required in the case of quaternization with volatile methyl halogenides, in particular methyl bromide. Methyl 10-camphorsulphonic and its single optical isomers are solid under normal conditions what simplifies technological operations, such as dosing, as well as storing thereof.

The process for the preparation of novel tiotropium salts with 10-camphorsulphonic acid and with single optical isomers thereof as well as monohydrates of the salts according to the present invention allows to obtain the desired product as stable, non-hygroscopic crystals, with high yield and with purity acceptable for being used in pharmaceutical compositions. Stability of crystalline anhydrous tiotropium salts with 1R-(−)-10-camphorsulphonic and 1S-(+)-10-camphorsulphonic acids was confirmed experimentally. Recrystallization of crystalline anhydrous tiotropium salts with 1R-(−)-10-camphorsulphonic and 1S-(+)-10-camphorsulphonic acids obtained as described above, from various organic solvents gave the products in its initial crystalline form, as shown by XRPD analysis. Therefore, no transformation or modification of a crystalline salt takes place.

Crystalline monohydrates of tiotropium salts with 1R-(−)-10-camphorsulphonic and 1S-(+)-10-camphorsulphonic also proved to be non-hygroscopic and stable.

In a further aspect, the present invention relates also to pharmaceutical compositions containing tiotropium salt with 10-camphorsulphonic acid or with single optical isomers thereof, or mixtures thereof, in combination with carrier(s) and/or pharmaceutical excipient(s).

Pharmaceutical compositions of the present invention are preferably in a unit dosage forms that may be manufactured using conventional methods known in the art. The methods include adding of an active ingredient to a carrier consisting of at least one substance that serves as a filler and/or an excipient.

The compositions containing novel tiotropium salts according to the present invention are preferably indicated for administration by inhalation. Compositions for inhalation comprise powder compositions, as well as compositions in a form of suspension and solution. The latter two compositions may contain a propellant, for instance chosen from fluorinated hydrocarbons. In order to improve physical stability of suspensions in a solvent, composition may contain surfactant, such as lecithin, oleic acid, sorbitan trioleate.

Dry powder compositions for inhalation preferably contain salt of tiotropium according to the present invention in a form of a powder and optionally excipients suitable for dry powder inhalation compositions, for example saccharides, such as monosaccharides, disaccharides, polysaccharides and sugar alcohols, such as arabinose, glucose, fructose, ribose, mannose, sucrose, trehalose, lactose, maltose, starches, dextran or mannitol. An especially preferred excipient is lactose, particularly in the form of the monohydrate. The dry powder may be used for filling of capsules made of gelatin or plastic for instance, or for filling of blisters, preferably made of laminated aluminum foil, for use in a dry powder inhalation device.

Particle size of the active ingredient suitable for the manufacturing of dry powder or of suspension for inhalation should be sufficiently small to be delivered to bronchi and lungs. Particle size optimal for that purpose should not exceed 100 μm, preferably 20 μm, and most preferably particle size of active ingredient should fall within the range 0.7-10 μm, preferably 1-5 μm. Powder particles or droplets with an aerodynamic diameter above 5 μm particularly do not reach lungs (or bronchii), and particles smaller than 1 μm are exhaled with air. Therefore, desired average particle size of active pharmaceutical ingredient or droplets of solution containing the active ingredient is in the range of 2-3 μm.

Pharmaceutical excipients serving as a carrier, where present, should have a maximum particle diameter of 300 μm, preferably of 200 μm. In a preferred embodiment an average particle size of a solid carrier falls within a range 40-100 μm, most preferably within the range 50-75 μm. Carrier may contain also an admixture of about 10% w/w of a fine fraction with an average particle size below 10 μm. Such an admixture, if necessary, prevents the tendency of active ingredient particles to agglomerate on a surface of coarse particles of a carrier.

If necessary, the active ingredient as well as a solid carrier may be milled to obtain particles with desired size distribution using conventional milling techniques, such as fluid bed jet milling, in a ball mill or in vibration mill.

Mechanical milling may result in partial amorphization of a crystalline substance. However, physical transformations of an active in form of a powder, are of no importance for compositions, where an active is completely dissolved.

Compositions in the form of solutions for inhalation may be formulated with water or a water/alcohol mixture as a solvent, with the addition of stabilizers, such as acids, buffers, chelating agents, and preservatives. Solutions may be subjected to sterilization by filtration or heating in autoclave. Solutions for inhalation are very convenient due to their higher dose uniformity. While physical stability of an active in solution does not present a problem, contact of the active with a solvent may adversely affect its chemical stability.

It is known from the prior art that tiotropium salts are susceptible to hydrolysis due to the presence of ester and epoxy functionalities. Addition of acidic stabilizers, such as organic or inorganic acids, to liquid compositions is thus necessary to ensure pH of the solution in the range of 2.5-4.5. Such compositions are disclosed in WO 98/027959, WO 02/36104, WO 03/084519 and WO 04/054580.

It has been unexpectedly found that novel salts according to the present invention are extremely stable under acidic conditions, what is reflected in the stability of liquid compositions. Increase in degradation products is significantly slower for novel salts of the invention than for tiotropium bromide.

Compositions for inhalation may be administered depending on a form thereof using powder inhalers, pressured metered dose inhalers, nebulizers.

Salts of tiotropium with 10-camphorsulphonic acid or with its single optical isomers are useful for the treatment of respiratory diseases, where the use of an anticholinergic is recommended, such as chronic obstructive pulmonary disease (COPD) and asthma.

The present invention relates also to the use of salts according to the invention for the treatment of respiratory tract diseases, in which administration of an anticholinergic is recommended, such as chronic obstructive pulmonary disease and asthma.

Analytical Methods

XRPD patterns were obtained using Rigaku MiniFlex X-ray diffractometer, with Cu Kα radiation, filter Kβ. Lamps parameters were set at 30 kV and 15 mA. Divergence and scattering slits were set at 4.2°, and a receiving slit was set at 0.3 mm. Deflection of rays was detected with scintillation detector NaI. The θ/2θ continuous scan at 1°/min with a step of 0.02° from 2.0 to 40° 2θ was used. The samples were prepared by pressing the solid in a quartz holder.

Chemical purity was determined by HPLC: Alliance 2695 chromatograph (Waters), detection UV, column ACE 3 C8 150×4.6 mm.

DSC (Differential Scanning calorimetry) analysis was carried out using the Mettler Toledo DSC 822 in an opened aluminum crucible, in temperature range 40-290° C., with heating rate 10° C./min.

NMR spectra were recorded on a Varian INNOVA-500 spectrometer.

Optical rotation was measured using Perkin Elmer 341 polarimeter.

### EXAMPLES

9-Methyl-3-oxa-9-azatricyclo[3.3.1.0^{2,4}]non-7-yl hydroxy(di-2-thienyl) acetate may be obtained by methods described in WO 91/04252.

### Example 1

### Preparation of methyl 1S-(+)-10-camphorsulphonate

The title compound was obtained according to J. I. Truillo, A. S. Gopalan; Tetrahedron Letters, Vol. 34, No. 46, pp. 7355-7358, 1993.

1S-(+)-10-Camphorsulphonic acid (0.1 mol) was suspended in 230 ml of methylene chloride. Then trimethyl orthoacetate (0.5 mol) was added dropwise. Reaction was carried out for 30 minutes at room temperature. Then the solvent was removed by evaporation, and the residue was triturated in heptane. Obtained crystals were filtered off and dried. Yield: 87%.

### Example 2

### Preparation of methyl 1R-(−)-10-camphorsulphonate

The title compound was obtained according to J. I. Truillo, A. S. Gopalan; Tetrahedron Letters, Vol. 34, No. 46, pp. 7355-7358, 1993.

1R-(−)-10-Camphorsulphonic acid (0.1 mol) was suspended in 230 ml of methylene chloride. Then trimethyl orthoacetate (0.5 mol) was added dropwise. Reaction was carried out for 30 minutes at room temperature. Then the solvent was removed by evaporation, and the residue was triturated in heptane. Obtained crystals were filtered off and dried. Yield: 85%.

### Example 3

### Preparation of methyl (±)-10-camphorsulphonate

The title compound was obtained according to J. I. Truillo, A. S. Gopalan; Tetrahedron Letters, Vol. 34, No. 46, pp. 7355-7358, 1993.

(±)-10-Camphorsulphonic acid (0.1 mol) was suspended in 230 ml of methylene chloride. Then trimethyl orthoacetate (0.5 mol) was added dropwise. Reaction was carried out for 30 minutes at room temperature. Then the solvent was removed by evaporation, and the residue was triturated in heptane. Obtained crystals were filtered off and dried. Yield: 89%.

### Example 4

### Tiotropium salt with 1R-(−)-10-camphorsulphonic acid (anhydrous)

9-Methyl-3-oxa-9-azatricyclo[3.3.1.0^{2,4}]non-7-yl hydroxy(di-2-thienyl) acetate (0.1 mol) was suspended in a methylene chloride/acetonitrile mixture (76 ml: 115 ml; 1:1.5 v/v). To the mixture methyl 1R-(−)-10-camphorsulphonate (0.5 mol) in 73 ml of acetonitrile was added. Reaction was carried out for 24 h at room temperature. Then the reaction mixture was concentrated by evaporation until crystals precipitated and the obtained crystals were triturated in methylene chloride, filtered off and dried in vacuum dryer at 30° C. Yield: 80%, HPLC purity: 99.74%.

[α]_{D}²⁰ (methanol, c=1) −14.89 ±0.77

Elemental analysis: C₂₉H₃₇NO₈S₃ (FW=623.8)

Calculated: C 55.84%; H 5.98%; N 2.25%; S 15.42%

Found: C 55.75%; H 6.09%; N 2.27%; S 15.25%.

¹H NMR (500 MHz) DMSO-d₆ 8 0.74 (s, 3H), 1.05 (s, 3H), 1.28 (q, 2H), 1.79 (d, 2H), 1.81-1.93 (m, 3H), 2.25 (dt, 1H), 2.37 (d, 1H), 2.68-2.84 (m, 3H), 2.87 (d, 2H), 3.04 (s, 3H), 3.24 (s, 3H), 3.50 (s, 2H), 4.12 (d, 2H), 5.12 (t, 1H), 7.01 (t, 2H), 7.13 (t, 2H), 7.43 (s, 1H), 7.53 (t, 2H).

¹³C NMR (500 MHz) DMSO-d₆ δ (ppm): 20.29, 20.88, 24.82, 27.12, 28.99, 42.82, 42.96, 47.34, 54.36, 56.26, 58.96, 64.43, 65.17, 77.01, 126.51, 126.88, 127.50, 147.34, 170.44, 217.12.

FIG. 1 shows XRPD pattern of obtained salt and Table 1 presents a detailed list of peaks with their relative intensities.

TABLE 1
Characteristic peaks, interplanar spacings d and relative intensities
of diffraction lines in the XRPD pattern for crystalline anhydrous
tiotropium salt with 1R-(−)-10-camphorsulphonic acid

| 2θ (±0.2° θ) | d (Å) | I/Iₒ |
|---|---|---|
| 5.4 | 16.29 | 100 |
| 9.7 | 9.11 | 12 |
| 10.8 | 8.15 | 19 |
| 14.1 | 6.27 | 13 |
| 14.3 | 6.17 | 12 |
| 16.3 | 5.44 | 26 |
| 16.9 | 5.25 | 21 |
| 17.1 | 5.14 | 24 |
| 17.2 | 5.15 | 23 |
| 18.3 | 4.85 | 17 |
| 18.7 | 4.74 | 25 |
| 20.5 | 4.32 | 11 |
| 20.7 | 4.28 | 16 |
| 21.2 | 4.18 | 21 |
| 21.8 | 4.08 | 12 |
| 22.4 | 3.97 | 33 |
| 26.3 | 3.38 | 15 |

Crystalline anhydrous tiotropium salt with 1R-(−)-10-camphorsulphonic acid shows on DSC diagram a single sharp endothermic signal in the temperature range of 220-230° C., that corresponds to melting of the salt.

### Example 5

### Tiotropium salt with 1R-(−)-10-camphorsulphonic acid (anhydrous)

9-Methyl-3-oxa-9-azatricyclo[3.3.1.0^{2,4}]non-7-yl hydroxy(di-2-thienyl) acetate (0.1 mol) was dissolved in ethanol (100 ml). To the mixture methyl 1R-(−)-10-camphorsulphonate (0.3 mol) was added. Reaction was carried out for 48 h at room temperature. Then the reaction mixture was concentrated by evaporation until crystals precipitated and the obtained crystals were triturated in ethanol, is filtered off and dried in vacuum dryer at 30° C. Yield: 68%, HPLC purity: 99.57%.

### Example 6

### Tiotropium salt with 1R-(−)-10-camphorsulphonic acid (anhydrous)

To 9-methyl-3-oxa-9-azatricyclo[3.3.1.0^{2,4}]non-7-yl hydroxy(di-2-thienyl) acetate (0.1 mol) in n-butanol (100 ml) methyl 1R-(−)-10-camphorsulphonate (0.5 mol) was added. Reaction was carried out for 48 h at room temperature. Then the reaction mixture was concentrated by evaporation until crystals precipitated and the obtained crystals were triturated in methylene chloride, filtered off and dried in vacuum dryer at 30° C. Yield: 71%, HPLC purity: 99.52%.

### Example 7

### Tiotropium salt with 1R-(−)-10-camphorsulphonic acid (anhydrous)

To 9-methyl-3-oxa-9-azatricyclo[3.3.1.0^{2,4}]non-7-yl hydroxy(di-2-thienyl) acetate (0.1 mol) in toluene (100 ml) methyl 1R-(−)-10-camphorsulphonate (0.5 mol) was added. Reaction was carried out for 48 h at room temperature. Then the reaction mixture was concentrated by evaporation until crystals precipitated and the obtained crystals were triturated in methylene chloride, filtered off and dried in vacuum dryer at 30° C. Yield: 71%, HPLC purity: 99.52%.

### Example 8

### Tiotropium salt with 1S-(+)-10-camphorsulphonic acid

9-Methyl-3-oxa-9-azatricyclo[3.3.1.0^{2,4}]non-7-yl hydroxy(di-2-thienyl) acetate (0.1 mol) was suspended in a mixture of methylene chloride/acetonitrile (76 ml: 115 ml; 1:1.5 v/v). To the mixture methyl 1S-(+)-10-camphorsulphonate (0.5 mol) in 73 ml of acetonitrile was added. Reaction was carried out for 24 h at room temperature. Then the reaction mixture was concentrated by evaporation until crystals precipitated and the obtained crystals were triturated in methylene chloride, filtered off and dried in vacuum dryer at 30° C. Yield: 70%, HPLC purity: 99.80%.

[α]_{D}²⁰ (methanol, c=1) 15.81±0.36

Elemental analysis: C₂₉H₃₇NO₈S₃ (FW=623.8)

calculated: C 55.85%; H 5.98%; N 2.25%; S 15.42%;

found: C 55.48%; H 5.90%; N 2.16%; S 15.65%.

¹³C NMR (500 MHz) DMSO-d₆ δ (ppm): 19.55, 20.14, 24.12, 26.39, 28.27, 42.12, 42.24, 46.64, 47.00, 53.63, 55.56, 58.24, 63.70, 76.30, 125.77, 126.13, 126.75, 146.64, 169.72, 216. 35.

¹H NMR (500 MHz) DMSO-d₆ δ (ppm): 0.74 (s, 3H), 1.05 (s, 3H), 1.26 (q, 2H), 1.77 (d, 2H), 1.81-1.94 (m, 3H), 2.24 (dt, 1H), 2.37 (d, 1H), 2.65-2.72 (m, 3H), 2.86 (d, 2H), 3.05 (s, 3H), 3.25 (s, 3H), 3.51 (s, 2H), 4.13 (d, 2H), 5.12 (t, 1H), 7.01 (t, 2H), 7.13 (t, 2H), 7.39 (s, 1H), 7.51 (t, 2H).

FIG. 2 shows XRPD pattern of the salt obtained above and Table 2 presents a detailed list of peaks with their relative intensities.

TABLE 2
Characteristic peaks, interplanar spacings and relative intensities
of diffraction lines in the XRPD pattern for crystalline anhydrous
tiotropium salt with 1S-(+)-10-camphorsulphonic acid

| 2θ (±0.2° θ) | d (Å) | I/Io |
|---|---|---|
| 5.6 | 15.88 | 100 |
| 9.8 | 9.02 | 14 |
| 11.0 | 8.07 | 16 |
| 14.2 | 6.22 | 16 |
| 14.5 | 6.12 | 14 |
| 16.4 | 5.40 | 22 |
| 17.0 | 5.20 | 22 |
| 17.2 | 5.15 | 30 |
| 17.3 | 5.11 | 31 |
| 18.4 | 4.81 | 22 |
| 18.9 | 4.70 | 25 |
| 20.7 | 4.28 | 16 |
| 20.9 | 4.25 | 18 |
| 21.3 | 4.16 | 25 |
| 21.9 | 4.06 | 10 |
| 22.5 | 3.95 | 26 |
| 26.4 | 3.37 | 17 |

Crystalline anhydrous tiotropium salt with 1S-(+)-10-camphorsulphonic acid shows on DSC diagram a single sharp endothermic signal in a temperature range of 220-230° C., corresponding to melting of the salt.

### Example 9

### Tiotropium salt with 1S-(+)-10-camphorsulphonic acid (anhydrous)

To 9-methyl-3-oxa-9-azatricyclo[3.3.1.0^{2,4}]non-7-yl hydroxy(di-2-thienyl) is acetate (0.1 mol) in ethanol (100 ml) methyl 1S-(+)-10-camphorsulphonate (0.3 mol) was added. Reaction was carried out for 48 h at room temperature. Then the reaction mixture was cooled in an ice bath, crystals thus obtained were filtered off, washed with chilled ethanol and dried in vacuum dryer at 30° C. Yield: 63%, HPLC purity: 99.41%.

### Example 10

### Tiotropium salt with 1S-(+)-10-camphorsulphonic acid (anhydrous)

To 9-methyl-3-oxa-9-azatricyclo[3.3.1.0^{2,4}]non-7-yl hydroxy(di-2-thienyl) acetate (0.1 mol) in n-butanol (100 ml) methyl 1S-(+)-10-camphorsulphonate (0.3 mol) was added. Reaction was carried out for 48 h at room temperature. Then the reaction mixture was concentrated by evaporation until crystals precipitated and the obtained crystals were triturated in methylene chloride, filtered off and dried in vacuum dryer at 30° C. Yield: 54%, HPLC purity: 99.53%.

### Example 11

### Tiotropium salt with 1S-(+)-10-camphorsulphonic acid (anhydrous)

To 9-methyl-3-oxa-9-azatricyclo[3.3.1.0^{2,4}]non-7-yl hydroxy(di-2-thienyl)-acetate (0.1 mol) in toluene (100 ml) methyl 1S-(+)-10-camphorsulphonate (0.4 mol) was added. Reaction was carried out for 48 h at room temperature. Then the reaction mixture was concentrated by evaporation until crystals precipitated and the obtained crystals were triturated in methylene chloride, filtered off and dried in vacuum dryer at 30° C. Yield: 47%, HPLC purity: 98.87%.

### Example 12

### Tiotropium salt with (±)-10-camphorsulphonic acid (anhydrous)

9-Methyl-3-oxa-9-azatricyclo[3.3.1.0^{2,4}]non-7-yl hydroxy(di-2-thienyl) acetate (0.1 mol) was suspended in a mixture of methylene chloride/acetonitrile (76 ml: 115 ml; 1:1.5 v/v). To the mixture methyl (±)-10-camphorsulphonate (0.5 mol) in 73 ml of acetonitrile was added. Reaction was carried out for 24 h at room temperature. Then the reaction mixture was concentrated by evaporation until crystals precipitated and the obtained crystals were triturated in acetone, filtered off and dried in vacuum dryer at 30° C. Yield: 80%, HPLC purity: 99.70%.

FIG. 3 shows XRPD pattern of the salt obtained above and Table 3 presents a detailed list of peaks with their relative intensities.

TABLE 3
Characteristic peaks, interplanar spacings and relative intensities
of diffraction lines in the XRPD pattern for crystalline tiotropium
salt with (±)-10-camphorsulphonic acid

| 2θ (±0.2° θ) | d (Å) | I/Iₒ |
|---|---|---|
| 5.4 | 16.47 | 100 |
| 10.5 | 8.40 | 16 |
| 15.4 | 5.74 | 24 |
| 15.7 | 5.64 | 16 |
| 16.5 | 5.36 | 17 |
| 18.6 | 4.77 | 16 |
| 20.7 | 4.28 | 25 |
| 23.4 | 3.80 | 19 |

### Example 13

### Crystalline monohydrate of tiotropium salt with 1R-(−)-10-camphorsulphonic acid

9-Methyl-3-oxa-9-azatricyclo[3.3.1.0^{2,4}]non-7-yl hydroxy(di-2-thienyl)acetate (0.1 mol) was suspended in a methylene chloride/acetonitrile mixture (76 ml: 115 ml; 1:1.5 v/v). To the mixture methyl 1R-(−)-10-camphorsulphonate (0.3 mol) in 73 ml of acetonitrile was added. Reaction was carried out for 4 h at 65° C. Then the reaction mixture was concentrated by evaporation until crystals precipitated, the obtained crystals were triturated in acetone and filtered off. Subsequently, crystals were suspended in water (100 ml), heated to 75° C. and maintained at that temperature for 10 minutes. The solution was then allowed to cool to room temperature. Obtained suspension of crystals was cooled in an ice bath to a temperature of about 10° C., stirred at that temperature for 1.5 h, and then product was filtered off and dried in vacuum at 40° C. Yield: 78%, HPLC purity: 99.88%.

[α]_{D}²⁰ (methanol, c=1) −14.21±0.80

Water content (KF): 2.9%

Elemental analysis:

C₂₉H₃₇NO₈S₃×1H₂O (FW=641.8)

calculated: C 54.27%; H 6.12%; N 2.18%; S 14.99%

found: C 54.29%; H 6.12%; N 2.24%; S 15.01%.

FIG. 4 shows XRPD pattern of the crystalline monohydrate obtained above and Table 4 presents a detailed list of peaks with their relative intensities.

TABLE 4
Characteristic peaks, interplanar spacings and relative intensities
of diffraction lines in the XRPD pattern for crystalline monohydrate
of tiotropium salt with 1R-(−)-10-camphorsulphonic acid

| 2θ (±0.2° θ) | d (Å) | I/Iₒ |
|---|---|---|
| 8.8 | 10.10 | 56 |
| 15.4 | 5.76 | 67 |
| 16.2 | 5.46 | 42 |
| 16.8 | 5.26 | 76 |
| 17.5 | 5.08 | 75 |
| 17.8 | 4.99 | 18 |
| 18.6 | 4.76 | 28 |
| 19.0 | 4.67 | 21 |
| 19.2 | 4.61 | 26 |
| 20.3 | 4.38 | 27 |
| 21.3 | 4.18 | 30 |
| 21.8 | 4.07 | 52 |
| 22.7 | 3.92 | 19 |
| 25.4 | 3.50 | 100 |
| 26.7 | 3.34 | 21 |
| 27.6 | 3.23 | 63 |
| 28.8 | 3.10 | 28 |
| 29.1 | 3.07 | 23 |
| 30.8 | 2.90 | 25 |
| 31.4 | 2.84 | 22 |
| 31.9 | 2.80 | 23 |
| 34.4 | 2.61 | 20 |
| 34.6 | 2.59 | 29 |
| 35.3 | 2.54 | 36 |

Crystalline monohydrate of tiotropium salt with 1R-(−)-10-camphorsulphonic acid shows on DSC diagram two endothermic signals: the first, relatively broad, in a temperature range of 105-130° C., and the second, sharp, in the range of 220-230° C. The first peak is attributed to melting of the hydrate, dehydration and transformation into anhydrous form. The second peak is attributed to melting of the anhydrous salt.

### Example 14

### Crystalline monohydrate of tiotropium salt with 1S-(+)-10-camphorsulphonic acid

9-Methyl-3-oxa-9-azatricyclo[3.3.1.0^{2,4}]non-7-yl hydroxy(di-2-thienyl) acetate (0.1 mol) was suspended in a mixture of methylene chloride/acetonitrile (76 ml: 115 ml; 1:1.5 v/v). To the mixture methyl 1R-(−)-10-camphorsulphonate (0.3 mol) in 73 ml of acetonitrile was added. Reaction was carried out for 4 h at 65° C. Then the reaction mixture was concentrated by evaporation until crystals precipitated, the obtained crystals were triturated in acetone and filtered off. Crystals were next suspended in water (100 ml), heated to 75° C. and maintained at that temperature for 10 minutes. Subsequently the solution was left to spontaneous cooling to room temperature. Obtained suspension of crystals was cooled in an ice bath to a temperature of about 10° C., stirred at that temperature for 1.5 h, and then the product was filtered off and dried in vacuum dryer at 40° C. Yield: 75%, HPLC purity: 99.92%.

[α]_{D}²⁰ (methanol, c=1) 14.76

Water content (KF): 2.8%

Elemental analysis:

C₂₉H₃₇NO₈S₃×1H₂O (FW=641.8)

calculated: C 54.27%; H 6.12%; N 2.18%; S 14.99%

found: C 54.26%; H 6.01%; N 2.15%; S 15.09%.

FIG. 5 shows XRPD pattern of the crystalline monohydrate obtained above and Table 5 presents a detailed list of peaks with their relative intensities.

TABLE 5
Characteristic peaks, interplanar spacings and relative intensities
of diffraction lines in the XRPD pattern for crystalline monohydrate
of tiotropium salt with 1S-(+)-10-camphorsulphonic acid

| 2θ (±0.2° θ) | d (Å) | I/Iₒ |
|---|---|---|
| 9.0 | 9.84 | 72 |
| 15.6 | 5.68 | 64 |
| 16.5 | 5.38 | 49 |
| 17.0 | 5.21 | 72 |
| 17.7 | 5.01 | 82 |
| 18.0 | 4.93 | 21 |
| 18.8 | 4.72 | 31 |
| 19.2 | 4.63 | 24 |
| 19.5 | 4.55 | 28 |
| 20.5 | 4.32 | 26 |
| 21.5 | 4.13 | 33 |
| 22.0 | 4.03 | 57 |
| 22.9 | 3.89 | 23 |
| 25.6 | 3.47 | 100 |
| 26.8 | 3.32 | 36 |
| 27.8 | 3.21 | 68 |
| 29.0 | 3.08 | 33 |
| 29.3 | 3.04 | 20 |
| 31.0 | 2.88 | 24 |
| 31.6 | 2.82 | 34 |
| 32.0 | 2.79 | 22 |
| 34.6 | 2.59 | 24 |
| 34.9 | 2.57 | 28 |
| 35.6 | 2.52 | 42 |

Crystalline monohydrate of tiotropium salt with 1S-(+)-10-camphorsulphonic acid shows on DSC diagram two endothermic signals: the first, relatively broad, in a temperature range of 105-130° C., and the second, sharp, in the range 220-230° C. The first peak corresponds to melting of the hydrate, dehydration and transformation into anhydrous form. The second peak corresponds to melting of the anhydrous salt.

### Example 15

### Stability of salts according to the invention under stress conditions—comparative experiments

Tiotropium salt with 1R-(−)-10-camphorsulphonic acid of the invention, prepared according to Example 4, crystalline monohydrate of tiotropium salt with 1R-(−)-10-camphorsulphonic acid of the invention, prepared according to Example 13, crystalline anhydrous tiotropium salt with 1S-(+)-10-camphorsulphonic acid of the invention, prepared according to Example 8, crystalline monohydrate of tiotropium salt with 1S-(+)-10-camphorsulphonic acid of the invention, prepared according to Example 14, and monohydrate of tiotropium bromide prepared according to Example of WO 02/30928 (tiotropium bromide was obtained according to Example 4 of WO 91/04252) were subjected to stability tests under stress conditions.

Stability Under Acidic Conditions

10 mg of each salt were separately weighed into a volumetric flask (10 ml) and 0.5 ml of 20% HCl aq was added. Obtained solutions were left for 15 min at 25° C. Then solutions were neutralized using 20% NaOH aq and a mixture of acetonitrile/water 1:1 (v/v) was added up to 10 ml. Purity was determined using HPLC method.

Stability Under Alkaline Conditions

10 mg of each salt were separately weighed into a volumetric flask (10 ml) and 0.5 ml of 0.01M NaOH aq was added. Obtained solutions were left for 5 min at 25° C. Then solutions were neutralized using 0.01M HCl aq and a mixture of acetonitrile/water 1:1 (v/v) was added up to 10 ml. Purity was determined using HPLC method.

Stability Under Oxidizing Conditions

10 mg of each salt were separately weighed into a volumetric flask (10 ml) and 0.5 ml of H₂O₂ was added. Obtained solutions were left for 2 h at 25° C. Then to the solutions a mixture of acetonitrile/water 1:1 (v/v) was added up to 10 ml. Purity was determined using HPLC method.

Stability Under the Influence of Moisture and Heat

100 mg of each salt were placed separately on Petri dishes in an desiccator equipped with a beaker containing a saturated NaCl solution. The desiccator was placed in an oven at 65° C. After 18 days samples were analyzed using HPLC method.

Photostability

20 mg of each salt were placed separately on Petri dishes and exposed to UV radiation (254 nm). After 14 days samples were analyzed using HPLC method.

Results of the tests are presented in Table 6.

Results of the above tests show an exceptional stability of tiotropium salts with 1R-(−)-10-camphorsulphonic acid and 1S-(+)-10-camphorsulphonic acid. The results of the tests under acidic conditions, under the influence of moisture and heat and photostability are of particular importance, since both an active substance and a pharmaceutical composition can most often be exposed to these conditions during manufacturing and storage. Excellent stability of the salts according to the present invention under these conditions makes them very suitable for industrial application.

TABLE 6
Results of stability tests under stress conditions

| | | Tiotropium salt with | Tiotropium salt with | Tiotropium salt with | Tiotropium salt with |
|---|---|---|---|---|---|
| | Tiotropium | 1R-(−)-10- | 1R-(−)-10- | 1S-(+)-10- | 1S-(+)-10- |
| Test | bromide, | camphorsul-phonic | camphorsul-phonic | camphorsul-phonic | camphorsul-phonic |
| condition | Monohydrate | acid, anhydrous | acid, monohydrate | acid, anhydrous | acid, monohydrate |
| Start | 98.97 | 99.74 | 99.88 | 99.80 | 99.92 |
| Acidic | 89.12 | 95.65 | 94.05 | 97.69 | 97.31 |
| Alkaline | 84.70 | 81.29 | 82.31 | 87.92 | 85.44 |
| Oxidizing | 95.44 | 99.12 | 99.43 | 99.76 | 99.84 |
| Moisture, | 94.61 | 99.14 | 98.97 | 98.24 | 99.84 |
| Heat | | | | | |
| UV radiation | 93.29 | 98.82 | 99.66 | 99.81 | 99.73 |

1. Salts of tiotropium with 10-camphorsulphonic acid, its single optical isomers or mixtures thereof

2. The salt according to claim 1 with 1S-(+)-10-camphorsulphonic acid.

3. The salt according to claim 1 with 1R-(−)-10-camphorsulphonic acid.

4. The salt according to claim 1 in a crystalline form.

5. The salt according to claim 2 in a crystalline anhydrous form having X-ray powder diffraction pattern (XRPD) with characteristic peaks at 2θ degrees 5.6, 16.4, 17.0, 17.2, 17.3, 18.9, 21.3, and 22.5 (±0.2° θ).

6. The salt according to claim 3 in a crystalline anhydrous form having X-ray powder diffraction pattern (XRPD) with characteristic peaks at 20 degrees 5.4, 16.3, 16.9, 17.1, 17.2, 18.7, 21.2, and 22.4 (±0.2° θ).

7. The salt according to claim 2 in a monohydrate form.

8. The salt according to claim 2 in a crystalline monohydrate form.

9. The salt according to claim 3 in a crystalline monohydrate form.

10. The salt according to claim 8 having an X-ray powder diffraction pattern (XRPD) with characteristic peaks at 2θ degrees 9.0, 15.6, 17.0, 17.7, 22.0, 25.6, and 27.8 (±0.2° θ).

11. The salt according to claim 9 having an X-ray powder diffraction pattern (XRPD) with characteristic peaks at 20 degrees 8.8, 15.4, 16.8, 17.5, 21.8, 25.4, and 27.6 (±0.2° θ).

12. A process for the preparation of a tiotropium salt with 10-camphorsulphonic acid or a single optical isomer thereof, which comprises quaternization of 9-methyl-3-oxa-9-azatricyclo[3.3.1.0^{2,4}]non-7-yl hydroxy(di-2-thienyl) acetate in an organic solvent selected from a group consisting of nitriles, alcohols, halogenated hydrocarbons, hydrocarbons, esters, ketones, ethers and mixtures thereof, with methyl 10-camphorsulphonate or a single optical isomer thereof, and then isolation of the tiotropium salt.

13. The process according to claim 12 wherein the tiotropium salt is isolated in a crystalline anhydrous form.

14. The process according to claim that wherein the quaternization is carried out in an acetonitrile/methylene chloride mixture.

15. The process according to claim 13 wherein the tiotropium salt is isolated by concentration of a reaction mixture to obtain a crystalline residue, followed by addition of an organic solvent to the crystalline residue, trituration, and finally filtration of the crystalline anhydrous tiotropium salt.

16. The process according to claim 15 wherein trituration is carried out in methylene chloride or acetone.

17. A process for the preparation of a crystalline monohydrate of tiotropium salt with 10-camphorsulphonic acid or a single optical isomer thereof characterized in that the salt prepared by the process as claimed in claim 12 is dissolved in water, preferably at elevated temperature, then the solution is cooled until a crystalline product precipitates and the crystalline product is isolated.

18. The process according to claim 17 wherein the salt is dissolved in water at a temperature in the range of 40-90° C., preferably at 70-80° C.

19. A pharmaceutical composition containing tiotropium salt with 10-camphorsulphonic acid as defined in claim 1, and at least one pharmaceutically acceptable carrier(s) and/or pharmaceutical excipient(s).

20. The method of treating respiratory diseases with tiotropium salts with 10-camphorsulphonic acid, which comprises administration to a patient in need of such treatment of a compound selected from claim 1 for the treatment of respiratory tract diseases.
